(19) United States
(12) Patent Application Publication
(10) Pub. No.: US 2007/0088031 A1
(43) Pub. Date: Apr. 19, 2007

(54) Novel chemical compounds

(76) Inventors: Masato Nakano, Ibaraki (JP); Yutaka Maeda, Ibaraki (JP)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/596,129

(22) PCT Filed: Nov. 17, 2004
(86) PCT No.: PCT/US04/38307

§ 371 Date: Jun. 1, 2006

Related U.S. Application Data
(60) Provisional application No. 60/526,811, filed on Dec. 4, 2003.

(51) Int. Cl. A61K 31/5377; A61K 31/519; C07D 491/02

(52) U.S. Cl. 514/234.2; 514/260.1; 544/118; 544/278

(57) ABSTRACT

The present invention relates generally to inhibitors of the kinases, such as GSK-3, and more particularly to fused pyrimidine compounds.

### FIELD OF THE INVENTION

The present invention relates generally to inhibitors of the kinases, such as GSK-3, and more particularly to fused pyrimidine compounds.

### BACKGROUND OF THE INVENTION

The present invention provides compounds that are useful pharmacological agents for disease states that are mediated (for example, alleviated) through the inhibition or antagonism, of protein kinases. In particular, the present invention relates to compounds that demonstrate protein tyrosine kinase and/or protein serine/threonine kinase inhibition.

The protein kinases represent a large family of proteins which play a central role in the regulation of a wide variety of cellular processes and maintaining control over cellular function (Hanks, et al., Science, 1988, 241, 42-52). The loss of control over cellular regulation can often lead to aberrant cell function or death, often resulting in a disease state in the parent organism. Inhibitors of certain kinases may also have utility in the treatment of diseases when the kinase is not misregulated, but is nonetheless essential for maintenance of the disease state. In this case, inhibition of the kinase activity would act either as a cure or palliative for these diseases.

GSK-3 (glycogen synthase kinase-3) is Identified as a kinase useful in the treatment of type II diabetes. GSK-3 inhibits glycogen synthase by direct phosphorylation. Upon insulin activation, GSK-3 is inactivated, thereby allowing the activation of glycogen synthase and possibly other insulin-dependent events.

Type II diabetes, otherwise known as Non-insulin Dependent Diabetes Mellitus (NIDDM), is initially characterized by decreased sensitivity to insulin (insulin resistance) and a compensatory elevation in circulating insulin concentrations. Increased insulin levels are caused by increased secretion from the pancreatic beta cells in an attempt to overcome the insulin resistance. The resulting hyperinsulinemia is associated with a variety of cardiovascular complications.

As insulin resistance worsens, the demand on the pancreatic beta cells steadily increases until the pancreas can no longer provide adequate levels of insulin, thereby resulting in elevated levels of glucose in the blood. Thus, diabetes causes impaired glucose transport into skeletal muscle and increased hepatic glucose production, in addition to inadequate insulin response. The disorders and conditions associated with hyperglycemia and hyperlipidemia include cardiovascular disease, renal failure, and blindness.

GSK-3 inhibition stimulates insulin-dependent processes and is consequently useful in the treatment of diseases and conditions, such as type II diabetes, that are mediated by GSK-3 activity, or, more specifically, characterized by a need for the inhibition of GSK-3.

For example, Klein et al., PNAS 93:8455-9 (1996) report that lithium ion inhibits GSK-3 activity. Lithium has been reported to have anti-diabetic effects such as reduction of plasma glucose levels, increased glycogen uptake, potentiation of insulin, and stimulation of glycogen synthesis in skin, muscle, and fat cells. Lithium, however, effects molecular targets other than GSK-3, and is, therefore, not a widely accepted therapy for diabetics.

Other examples of GSK-3 mediated diseases or conditions include, without limitation, obesity, various CNS disorders such as Alzheimer's Disease, bipolar disorder, and schizophrenia, neurotraumatic injuries such as acute stroke, immune potentiation, baldness or hair loss, atherosclerotic cardiovascular disease, hypertension, polycystic ovary syndrome, ischemia, brain trauma or injury, immunodeficiency, and cancer. See, for example, published PCT application WO 00/38675, the background of which is herein incorporated by reference.

Thus, the compounds of the present invention are believed useful is a variety of disease states, each of which may be characterized as mediated by inhibition or antagonism of protein kinases, more particularly GSK-3.

### SUMMARY OF THE INVENTION

In a first aspect, the present invention relates to a compound of the formula I, or a salt, solvate, or a physiologically functional derivative thereof 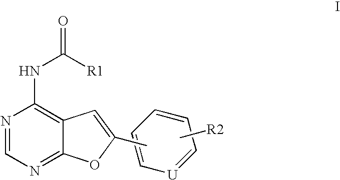
in which
U is CH or N; and
R1 is C₁₋₆alkyl, C₃₋₈cycloalkyl, —CH₂CH₂SCH₃, —CH₂—C₃₋₈cycloalkyl, phenyl optionally substituted with halogen or nitro; or
R1 is a radical of formula 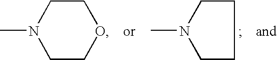
when U is CH, R2 is hydrogen, halogen, C₁₋₆alkyl, or —OCH₃; and
when U is N, R2 is hydrogen.
   In one preferred embodiment, a compound of formula I has the formula 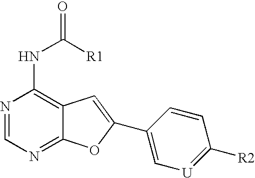
   in which R1, R2 and U are as define above.

Another aspect of the present invention inlcudes a method for the treatment or prophylaxis of a disorder in a mammal, said disorder being characterized by misregulation of GSK-3, comprising, administering to the mammal a therapeutically effective amount of a compound of the formula I or a salt, solvate, or a physiologically functional derivative thereof. Preferably the disorder is Type II Diabetes.

Another aspect of the present invention includes pharmaceutical compositions comprising a therapeutically effective amount of a compound of formula I, or a salt, solvate, or a physiologically functional derivative thereof and one or more of pharmaceutically acceptable carriers, diluents and excipients. Preferably the composition further includes at least one additional agent for the treatment or prophylaxis of diabetes.

Another aspect of the present invention includes a pharmaceutical composition that comprises a therapeutically effective amount of a compound of formula I, or a salt, solvate, or a physiologically functional derivative thereof, and one or more of pharmaceutically acceptable carriers, diluents and excipients for preventing or treating conditions mediated by GSK-3.

Another aspect of the present invention includes the use of a compound as herein described, or a salt, solvate, or a physiologically functional derivative thereof in the preparation of a medicament for use in the treatment of a disorder mediated by inappropriate GSK-3 activity. Preferably the disorder is Type II Diabetes.

Another aspect of the present invention includes a method of treating diabetes in a mammal, comprising administering to said mammal a therapeutically effective amount of a compound of formula I or salt, solvate or physiologically functional derivative thereof.

Another aspect of the present invention includes a method of treating diabetes in a mammal, comprising administering to said mammal therapeutically effective amounts of (i) a compound of formula I or salt, solvate or physiologically functional derivative thereof and (ii) at least one additional anti-diabetic therapy.

### DETAILED DESCRIPTION

The compounds of the present invention may be employed alone or in combination with other therapeutic agents for the treatment of GSK-3 mediated conditions. In particular, in type II diabetes treatment, combinations of the compounds of the present invention with other anti-diabetic agents is envisaged. Combination therapies according to the present invention include the administration of at least one compound of the present invention or salt, solvate, or physiologically functional derivative thereof, and the use of at least one other diabetic treatment method. Preferably, combination therapies according to the present invention comprise the administration of at least one compound of the present invention and at least one other pharmaceutically active agent, such as insulin, α-glucosidase inhibitors, biguanides, insulin secretagogues, or insulin sensitizers. Non-limiting examples of α-glucosidase inhibitors include acarbose, emiglitate, miglitol, and voglibose. Non-limiting examples of biguanides include metformin, buformin, and phenformin. Non-limiting examples of insulin secretagogues include sulphonylureas. Non-limiting examples of insulin sensitizers include peroxisome proliferator activated receptor (PPAR) ligands, such as PPAR-γ agonists, for example Actos™ and Avandia™. The compound(s) of the present invention and other pharmaceutically active agent(s) may be administered together or separately. When administered separately the administration may occur simultaneously or sequentially in any order. The amounts of the compound(s) of the present invention and the other pharmaceutically active agent(s) and the relative timings of administration will be selected in order to achieve the desired combined therapeutic effect.

The compounds of the present invention and at least one additional diabetic treatment therapy may be employed in combination concomitantly or sequentially in any therapeutically appropriate combination with such other anti-diabetic therapies. The administration in combination of a compound of the present invention with other anti-diabetic agents may be in combination in accordance with the invention by administration concomitantly in (1) a unitary pharmaceutical composition including both compounds or (2) separate pharmaceutical compositions each including one of the compounds. Alternatively, the combination may be administered separately in a sequential manner wherein one agent is administered first and the other second or vice versa. Such sequential administration may be close in time or remote in time.

The mammal requiring treatment with a compound of the present invention is typically a human being.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the numbering of the scaffolds in the formula I is assigned as shown in the structure followings:

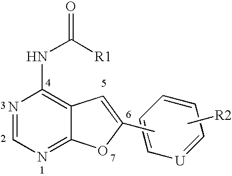

As used herein, the term “alkyl” refers to a straight or branched chain hydrocarbon. Furthermore, as used herein, the term “C₁₋₆ alkyl” refers to an alkyl group as defined above containing at least 1, and at most 6, carbon atoms. Examples of branched or straight chained “C₁₋₆ alkyl” groups useful in the present invention include methyl, ethyl, n-propyl, isopropyl, isobutyl, n-butyl,t-butyl, n-pentyl, n-hexyl, and the like.

As used herein, the term “halogen” refers to fluorine (F), chlorine (Cl), bromine (Br), or iodine (I).

As used herein, the term “C₃₋₈ cycloalkyl” refers to a non-aromatic cyclic hydrocarbon ring having from three to eight carbon atoms. Exemplary “C₃₋₈ cycloalkyl” groups include cyclopropyl, cyclobutyl, cyclopentyl, cyclohexyl, cycloheptyl, and cyclooctyl.

As used herein, the term “optionally” means that the subsequently described event(s) may or may not occur, and includes both event(s), which occur, and events that do not occur.

As used herein, the term “physiologically functional derivative” refers to any pharmaceutically acceptable derivative of a compound of the present invention, for example, an ester or an amide, which upon administration to a mammal is capable of providing (directly or indirectly) a compound of the present invention or an active metabolite thereof. Such derivatives are clear to those skilled in the art, without undue experimentation, and with reference to the teaching of Burger's Medicinal Chemistry And Drug Discovery, 5th Edition, Vol 1: Principles and Practice, which is incorporated herein by reference to the extent that it teaches physiologically functional derivatives.

As used herein, the term “solvate” refers to a complex of variable stoichiometry formed by a solute (in this invention, a compound of formula I or a salt or physiologically functional derivative thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

As used herein, the term “substituted” refers to substitution with the named substituent or substituents, multiple degrees of substitution being allowed unless otherwise stated.

Certain compounds described herein may contain one or more chiral atoms, or may otherwise be capable of existing as two enantiomers, or two or more diastereoisomers. Accordingly, the compounds of this invention include mixtures of enantiomers/diastereoisomers as well as purified enantiomers/diastereoisomers or enantiomerically/diastereoisomerically enriched mixtures. Also included within the scope of the invention are the individual isomers of the compounds represented by formula I above as well as any wholly or partially equilibrated mixtures thereof. The present invention also covers the individual isomers of the compounds represented by the formulas above as mixtures with isomers thereof in which one or more chiral centers are inverted. It should be understood that all tautomers and mixtures of tautomers are also included within the scope of the compounds of formula I.

Typically, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts derived from a nitrogen on a substituent in the compound of formula I. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this invention and these form a further aspect of the invention.

While it is possible that, for use in therapy, therapeutically effective amounts of a compound of formula I, as well as salts, solvates and physiological functional derivatives thereof, may be administered as the raw chemical, it is possible to present the active ingredient as a pharmaceutical composition. Accordingly, the invention further provides pharmaceutical compositions (otherwise referred to as pharmaceutical formulations), which include therapeutically effective amounts of compounds of the formula I and salts, solvates and physiological functional derivatives thereof, and one or more pharmaceutically acceptable carriers, diluents, or excipients. The compounds of the formula I and salts, solvates and physiological functional derivatives thereof, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the formula I or salts, solvates and physiological functional derivatives thereof, with one or more pharmaceutically acceptable carriers, diluents or excipients.

Pharmaceutical formulations may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the formula I, depending on the condition being treated, the route of administration and the age, weight and condition of the patient, or pharmaceutical formulations may be presented in unit dose forms containing a predetermined amount of active ingredient per unit dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken Into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The compounds of formula I, and salts, solvates and physiological functional derivatives thereof, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of formula I, and salts, solvates and physiological functional derivatives thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

A therapeutically effective amount of a compound of the present invention will depend upon a number of factors including, for example, the age and weight of the animal, the precise condition requiring treatment and its severity, the nature of the formulation, and the route of administration, and will ultimately be at the discretion of the attendant physician or veterinarian. However, an effective amount of a compound of formula I for GSK-3 mediated diseases or conditions including, without limitation, diabetes (in particular Type II Diabetes), obesity, various CNS disorders such as Alzheimer's Disease, bipolar disorder, and schizophrenia, neurotraumatic injuries such as acute stroke, immune potentiation, baldness or hair loss, atherosclerotic cardiovascular disease, hypertension, polycystic ovary syndrome, ischemia, brain trauma or injury, immunodeficiency, and cancer, will generally be in the range of 0.1 to 100 mg/kg body weight of recipient (mammal) per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a salt or solvate, or physiologically functional derivative thereof, may be determined as a proportion of the effective amount of the compound of formula I per se. It is envisaged that similar dosages would be appropriate for treatment of the other conditions referred to above.

Method of Preparation

Compounds of general formula I may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula I. Those skilled in the art will recognize if a stereocenter exists in compounds of formula I. Accordingly, the present invention includes both possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

More particularly, the compounds of the formula I can be made by the process of Scheme A or a variant thereof. Any person skilled in the art can readily adapt the process of A, such the stoichemistry of the reagents, temperature, solvents, etc. to optimize the yield of the products desired. 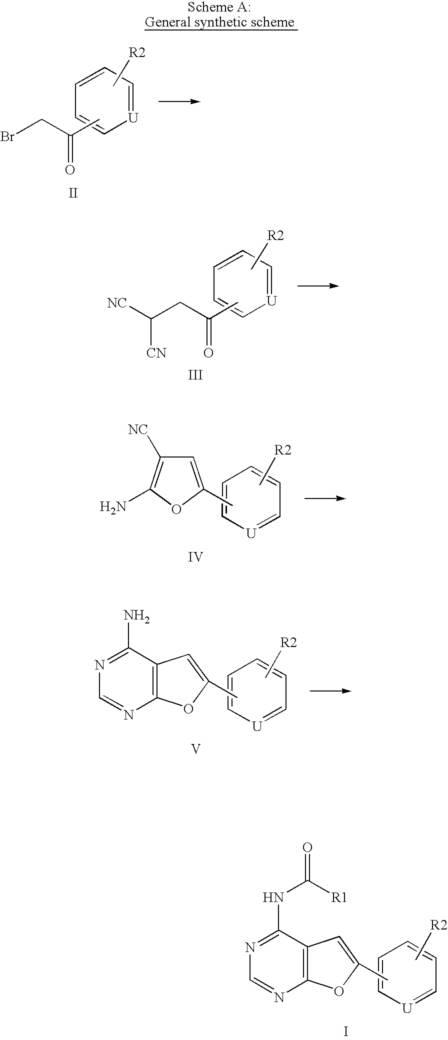
Briefly bromide of a compound of formula II is displaced with an anion made from manolonitrile and a base such as sodium ethoxide. A compound of formula III thus formed is then cyclized to afford a furan of formula IV under acidic or basic conditions. When U is CH in Scheme A, a compound of formula IV is subsequently converted into a compound of formula V under heating in the presence of formamide. When U is N in Scheme A, a compound of formula IV is subsequently converted into a compound of formula V by treating a compound of formula IV with diethoxymethyl acetate followed by cyclization with NH₃ gas. Finally a compound of formula V is acylated with a compound of formula R1(C═O)L, in which L is a typical leaving group such as chloride to afford a compound of formula I. In Scheme A, U, R1 and R2 are as defined previously.

Intermediates useful in the present invention include compounds of formula V: 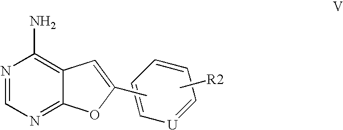
where
U is CH or N; and
when U is CH, R2 is hydrogen, halogen, C₁₋₆alkyl, or —OCH₃; or
when U is N, R2 is hydrogen.

Specific compounds of formula V useful in the synthesis of compounds of the present invention are:

6-(4-Methoxy-phenyl)-furo[2,3-d]pyrimidin-4-ylamine;

6-Phenyl-furo[2,3-d]pyrimidin-4-ylamine;

6-(4-Chloro-phenyl)-furo[2,3-d]pyrimidin-4-ylamine;

6-p-Tolyl-furo[2,3-d]pyrimidin-4-ylamine;

6-(4-Fluoro-phenyl)-furo[2,3-d]pyrimidin-4-ylamine; and

6-Pyridin-3-yl-furo[2,3-d]pyrimidin-4-ylamine.

Additional compounds useful in the synthesis of compounds of the present invention are:
Furo[2,3-d]pyrimidin-4-ylamine; and
6-Bromo-furo[2,3-d]pyrimidin-4-ylamine.

More detailed descriptions of Scheme A process appear in the Examples below.

### Specific Embodiments

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations rnay be used in the examples and throughout the specification:
g (grams);
L (liters);
μL (microliters);
M (molar);
i. v. (intravenous);
MHz (megahertz);
mmol (millimoles);
min (minutes);
mp (melting point);
Tr (retention time);
MeOH (methanol);
TEA (triethylamine);
TFAA (trifluoroacetic anhydride);
DMSO (dimethylsulfoxide);
DME (1,2-dimethoxyethane);
DCE (dichloroethane);
DMPU (N,N′-dimethylpropyleneurea);
mg (milligrams);
mL (milliliters);
psi (pounds per square inch);
mM (millimolar);
Hz (Hertz);
mol (moles);
rt (room temperature);
h (hours);
TLC (thin layer chromatography);
RP (reverse phase);
i-PrOH (isopropanol);
TFA (trifluoroacetic acid);
THF (tetrahydrofuran);
AcOEt (ethyl acetate);
DCM (dichloromethane);
DMF (N,N-dimethylformamide);
(CDI (1,1-carbonyldiimidazole);
IBCF (isobutyl chloroformate);
HOSu (N-hydroxysuccinimide);
mCPBA (meta-chloroperbenzoic acid;
BOC (tert-butyloxycarbonul);
FMOC (9-fluorenylmethoxycarbonyl);
DCC (dicyclohexylcarbodiimide);
CBZ (benzyloxycarbonyl);
Ac (acetyl);
TMSE (2-(trimethylsilyl)ethyl);
TIPS (triisopropylsilyl);
DMAP (4-dimethylaminopyridine);
ATP (adenosine triphosphate);
DMEM (Dulbecco's modified Eagle medium);
HPLC (high pressure liquid chromatography);
BOP (bis(2-oxo-3-oxazolidinyl)phosphinic chloride);
TBAF (tetra-n-butylammonium fluoride);
HBTU (O-Benzotriazole-1-yl-N,N,N′,N′-tetramethyluronium hexafluorophosphate);
HEPES (4-(2-hydroxyethyl)-1-piperazine ethane sulfonic acid);
DPPA (diphenylphosphoryl azide);
fHNO₃ (fumed HNO₃); and
EDTA (ethylenediaminetetraacetic acid).
HOAC (acetic acid);
HOBT (1-hydroxybenzotriazole);
EDC (ethylcarbodiimide hydrochloride
atm (atmosphere);
TMS (trimethylsilyl);
TBS (t-butyldimethylsilyl);
BSA (bovine serum albumin)
HRP (horseradish peroxidase);

All references to ether are to diethyl ether; brine refers to a saturated aqueous solution of NaCl. Unless otherwise indicated, all temperatures are expressed in °C. (degrees Centigrade). All reactions are conducted under an inert atmosphere at room temperature unless otherwise noted.

1H NMR spectra were recorded on a Varian VXR-300, a Varian Unity-300, a Varian Unity-400 instrument, a Brucker AVANCE-400, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), quint (quintet), m (multiplet), br (broad).

Low-resolution mass spectra (MS) were recorded on a JOEL JMS-AX505HA, JOEL SX-102, or a SCIEX-APliii spectrometer; LC-MS were recorded on a micromass 2MD and Waters 2690; high resolution MS were obtained using a JOEL SX-102A spectrometer. All mass spectra were taken under electrospray ionization (ESI), chemical ionization (CI), electron impact (EI) or by fast atom bombardment (FAB) methods. Infrared (IR) spectra were obtained on a Nicolet 510 FT-IR spectrometer using a 1-mm NaCl cell. Most of the reactions were monitored by thin-layer chromatography on 0.25 mm E. Merck silica gel plates (60F-254), visualized with UV light, 5% ethanolic phosphomolybdic acid or p-anisaldehyde solution. Flash column chromatography was performed on silica gel (230-400 mesh, Merck).

### Example 1

### Hexanoic acid [6-(4-methoxy-phenyl)-furo[2,3-d]pyrimidin-4-yl]-amide

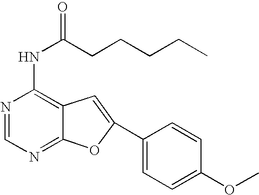

### Example 1

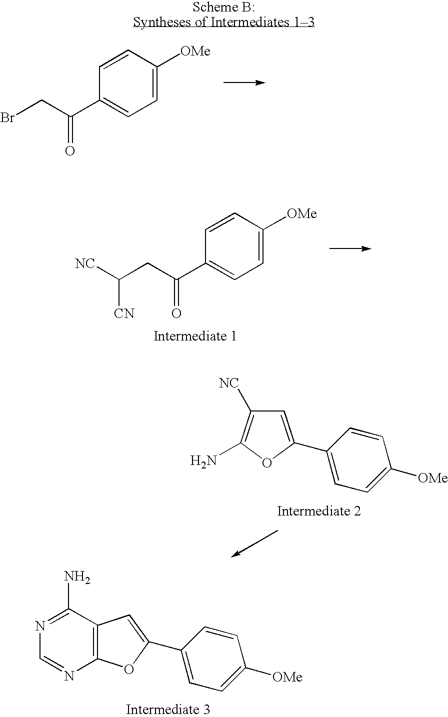 Intermediate 1:

### 2-[2-(4-Methoxy-phenyl)-2-oxo-ethyl]-malononitrile

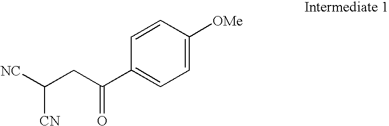

To a solution of sodium ethoxide (3.6 g, 53 mmol) in ethanol (60 ml) was added malononitrile (3.8 g, 57.5 mmol). The mixture was stirred at room temperature for 15 min, and then the mixture was chilled in an ice-water bath. To the mixture was added α-bromo-p-methoxyacetophenone (11.0 g, 48 mmol), and then stirred at room temperature for 1 hour. The resultant mixture was poured into cold water (120 ml). Precipitated materials were filtrated, washed with water, and dried under reduced pressure to afford Intermediate 1 (10.28 g) as a brown solid. 1H NMR (400 MHz, CDCl3) ppm 3.72(d, J=6.8 Hz, 2H), 3.92(s, 3H), 4.42(t, J=6.8 Hz, 1H), 6.98(m, 2H), 7.93(m, 2H).

Intermediate 2:

### 2-Amino-5-(4-methoxy-phenyl)-furan-3-carbonitrile

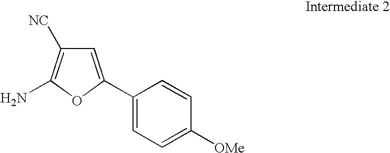

To a suspension of 2-[2-(4-methoxy-phenyl)-2-oxo-ethyl]-malononitrile (Intermediate 1, 9.66 g, 45.1 mmol) in acetic acid (50 ml) was added conc. hydrogen chloride (11, 3 ml). The mixture was stirred at room temperature for 2 hours, and then poured into water. The resultant precipitation was filtrated, washed with water and ethanol, and dried under reduced pressure to afford Intermediate 2 (5.54 g, 56%) as a solid. 1 H NMR (400 MHz, CDCl3) ppm 3.83(s, 3H), 4.74(brs, 2H), 6.39(s, 1H), 6.90(m, 2H), 7.42(m, 2H).

Intermediate 3:

### 6-(4-Methoxy-phenyl)-furo[2,3-d]pyrimidin-4-ylamine

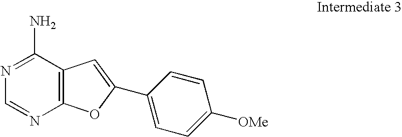

A solution of Intermediate 2 (5.54 g, 25.9 mmol) in formamide (100 ml) was heated at 200° C. for 1 hour. The mixture was cooled with an ice bath, and then poured into cold water. The precipitated material was filtrated, washed with water and ethanol, and dried under reduced pressure to give Intermediate 3 (5.61 g, 69%) as a solid. 1H NMR (400 MHz, CDCl3) ppm 3.87(s, 3H), 5.14(s, 2H), 6.72(s, 1H), 6.99(m, 2H), 7.78(m, 2H), 8.38(s, 1H). LC/MS: m/z 242 (M+1)⁺;

### Hexanoic acid [6-(4-methoxy-phenyl)-furo[2,3-d]pyrimidin-4-yl]-amide

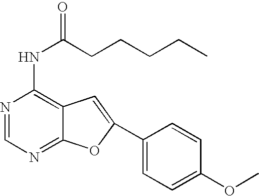

### Example 1

To a cooled solution of 6-(4-methoxy-phenyl)-furo[2,3-d]pyrimidin-4-ylamine (Intermediate 3, 53.0 mg, 0.22 mmol) in DMF (2.55 ml) was added sodium hydride (7.9 mg, 0.33 mmol) followed by hexanoyl chloride (33.8 μl, 0.24 mmol). The mixture was stirred at room temperature for 1 hour, and then poured into large amount of saturated ammonium chloride and the resulting solid was collected by filtration. This material was purified by chromatography on silica gel being eluted with 25% ethyl acetate-chloroform to give the title compound as a pale yellow solid (33%). 1H NMR (400 MHz, DMSO-d6) ppm 0.87-0.91(m, 3H), 1.30-1.34(m, 4H), 1.61-1.69(m, 2H), 3.84(s, 3H), 7.08-7.11 (m, 2H), 7.35(s, 1H), 7.85-7.87(m, 2H), 8.62(s, 1H), 11.07(s, 1H). LC/MS: m/z 340 (M+1)⁺, 341 (M+2)⁺, 342 (M+3)⁺, 338 (M−1)⁻, 339 (M+1)⁻.

Compounds of Examples 2-23 are made according to Scheme A or analgous to that of Example 1.

### Example 2

### N-[6-(4-Methoxy-phenyl)-furo[2,3-d]pyrimidin-4-yl]-isobutyramide

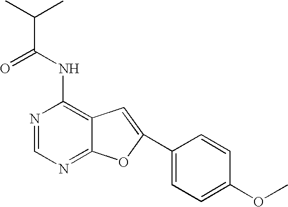

### EXAMPLE 2

1H NMR (400 MHz, DMSO-d6) ppm 1.17(s, 3H), 1.19(s, 3H), 2.84-2.94(m, 1H), 3.84(s, 3H), 7.08-7.10(m, 2H), 7.36(s, 1H), 7.85-7.88 (m, 2H), 8.63(s, 1H), 11.06(s, 1H). LC/MS: m/z 312(M+1)⁺, 313 (M+2)⁺, 310 (M−1)⁻, 311 (M+1)⁻.

### Example 3

### Cyclopentanecarboxylic acid [6-(4-methoxy-phenyl)-furo[2,3-d]pyrimidin-4-yl]-amide

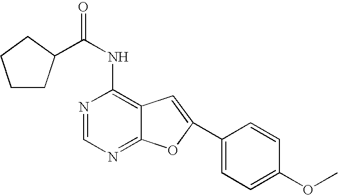

### Example 3

To a cooled solution of 6-(4-methoxy-phenyl)-furo[2,3-d]pyrimidin-4-ylamine (Intermediate 3, 50.0 mg, 0.21 mmol) in DMF (2.5 ml) was added sodium hydride (7.5 mg, 0.31 mmol) followed by cyclopenanecarbonyl chloride (28.7 μl, 0.23 mmol). The mixture was stirred at room temperature for 2 hours, and then poured into large amount of saturated ammonium chloride and the resulting solid collected by filtration. This material was purified by chromatography on silica gel being eluted with 9% ethyl acetate-chloroform to give the title compound as a pale yellow solid (54%). 1H NMR (400 MHz, DMSO-d6) ppm 1.54-1.63(m, 2H), 1.66-1.85(m, 4H), 1.91-1.99(m, 2H), 3.08(dt, J=8.1 Hz, 16.2 Hz), 3.84(s, 3H), 7.07-7.11(m, 2H), 7.35(s, 1H), 7.84-7.89(m, 2H), 8.62(s, 1H), 11.07(s, 1H). LC/MS: m/z 338 (M+1)⁺, 339 (M+2)⁺, 336 (M−1)⁻, 337 (M+1)⁻.

### Example 4

### N-[6-(4-Methoxy-phenyl)-furo[2,3-d]pyrimidin-4-yl]-3-methylsulfanyl-propionamide

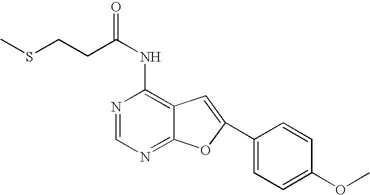

### Example 4

1H NMR (400 MHz, DMSO-d6) ppm 2.12(s, 3H), 2.78-2.86(m, 4H), 3.84(s, 3H), 7.08-7.11 (m, 2H), 7.36(s, 1H), 7.84-7.87(m, 2H), 8.63(s, 1H), 11.17(s, 1H). LC/MS: m/z 344 (M+1)⁺, 345 (M+2)⁺, 346 (M+3)⁺, 342 (M−1)⁻, 343 (M+1)⁻, 344 (M+2)⁻.

### Example 5

### 3-Fluoro-N-[6-(4-methoxy-phenyl)-furo[2,3-d]pyrimidin-4-yl]-benzamide

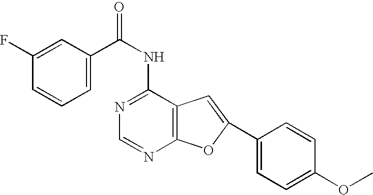

### Example 5

1H NMR (400 MHz, DMSO-d6) ppm 3.84(s, 3H), 7.08-7.13(m, 2H), 7.30(s, 1H), 7.53(dt, J=2.0 Hz, 8.6 Hz, 1H), 7.56-7.66(m, 1H), 7.89-7.97(m, 4H), 8.73(s, 1H), 11.62(s, 1H). LC/MS: m/z 364 (M+1)⁺, 365 (M+2)⁺, 366 (M+3)⁺, 362 (M−1)⁻, 363 (M+1)⁻, 364 (M+2)⁻.

### Example 6

### Cyclohexanecarboxylic acid [6-(4-methoxy-phenyl)-furo[2,3-d]pyrimidin-4-yl]-amide

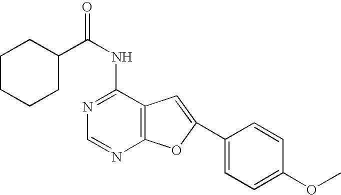

### Example 6

1H NMR (400 MHz, DMSO-d6) ppm 1.17-1.34(m, 3H), 1.41-1.50(m, 2H), 1.66(d, 11.1 Hz, 1H), 1.76-1.79(m, 2H), 1.89(d, 13.1 Hz), 2.59-2.67(m, 1H), 3.84(s, 3H), 7.08-7.10(m, 2H), 7.34(s, 1H), 7.85-7.89(m, 2H), 8.61(s, 1H) 11.00(s, 1H). LC/MS: m/z 352 (M+1)⁺, 353 (M+2)⁺, 354 (M+3)⁺, 350 (M−1)⁻, 351 (M+1)⁻.

### Example 7

### Cyclopropanecarboxylic acid [6-(4-methoxy-phenyl)-furo[2,3-d]pyrimidin-4-yl-amide

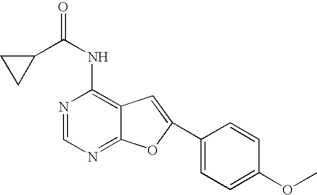

### Example 7

1H NMR (400 MHz, DMSO-d6) ppm 0.90-0.98(m, 4H), 2.11-2.18(m, 1H), 3.83(s, 3H), 7.07-7.11(m, 2H), 7.35(s, 1H), 7.83-7.88(m, 2H), 8.63(s, 1H), 11.42(s, 1H). LC/MS: m/z 310 (M+1)⁺, 311 (M+2)⁺, 308 (M−1)⁻, 309 (M+1)³¹ .

### Example 8

### Furan-2-carboxylic acid [6-(4-methoxy-phenyl)-furo[2,3-d]pyrimidin-4-yl]-amide

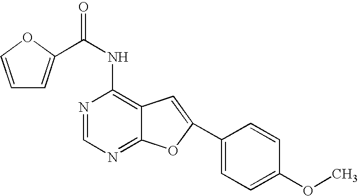

### Example 8

1H NMR (400 MHz, DMSO-d6) ppm 3.84(s, 3H), 6.77(dd, J=3.6 Hz, 1.6 Hz, 1H), 7.08-7.12(m, 2H), 7.32(s, 1H), 7.71(dd, J=3.6 Hz, 1.6Hz, 1H), 7.89-7.92(m, 2H), 8.06(s, 1H), 8.71(s, 1H), 11.41(s, 1H). LC/MS: m/z 336 (M+1)⁺, 337 (M+2)⁺.

### Example 9

### 2-Cyclopentyl-N-[6-(4-methoxy-phenyl)-furo[2,3-d]pyrimidin-4-yl]-acetamide

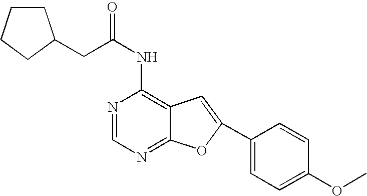

### Example 9

1H NMR (400 MHz, CDCl3-d6) ppm 1.22-1.31(m, 2H), 1.56-1.74(m, 4H), 1.91-1.99(m, 2H), 2.39(q, J=7.4 Hz, 1H), 2.53(d, J=7.4 Hz, 2H), 3.25(dt, J=10.4 Hz, 4.0 Hz, 1H), 3.87(s, 3H), 6.97-7.01(m, 2H), 7.41(s, 1H), 7.83-7.87(m, 2H), 8.06(s, 1H), 8.55(s, 1H). LC/MS: m/z 352 (M+1)⁺, 353 (M+2)⁺, 354 (M+3)⁺, 350 (M−1)⁻, 351 (M+1)⁻.

### Example 10

### N-[6-(4-Methoxy-phenyl)-furo[2,3-d]pyrimidin-4-yl]-3-nitro-benzamide

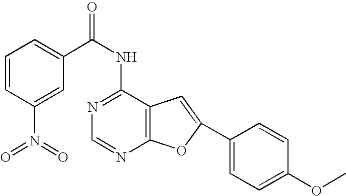

### Example 10

1H NMR (400 MHz, CDCl3-d6) ppm 3.89(s, 3H), 6.99-7.03(m, 2H), 7.43(s, 1H), 7.80(t, J=8.0 Hz, 1H), 7.87-7.91(m, 2H), 8.35(d, J=8.0 Hz, 1H), 8.51(d, J=8.0 Hz, 1H), 8.65(s, 1H), 8.82(s, 1H), 8.89(s, 1H). LC/MS: m/z 391 (M+1)⁺, 392 (M+2)⁺, 393 (M+3)⁺, 389 (M−1)⁻, 390 (M+1)⁻.

### Example 11

### N-[6-(4-Methoxy-phenyl)-furo[2,3-d]pyrimidin-4-yl]-4-nitro-benzamide

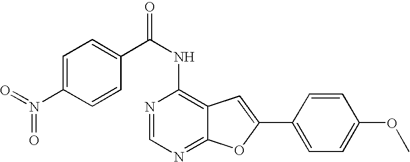

### Example 11

1H NMR (400 MHz, DMSO-d6) ppm 3.84(s, 3H), 7.09-7.11 (m, 2H), 7.33(s, 1H), 7.91(d, J=8.0 Hz, 2H), 8.30(d, J=8.0 Hz, 2H), 8.38-8.41(m, 2H), 8.75(s, 1H), 11.87(s, 1H). LC/MS: m/z 391 (M+1)⁺, 392 (M+2)⁺, 393 (M+3)⁺, 389 (M−1)⁻, 390 (M+1)⁻, 391 (M+2)⁻.

Intermediate 4:

### 6-Phenyl-furo[2,3-d]pyrimidin-4-ylamine

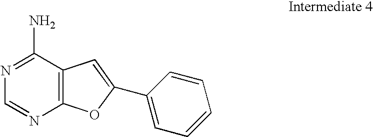

Prepared according to the similar methods as for Intermediate 3

1H NMR (400 MHz, CDCl3) ppm 5.19 (br, 2H), 6.87(s, 1H), 7.38(m, 1 H), 7.47(m, 2H), 7.85(m, 2H), 8.40(s, 1H). LC/MS: m/z 212 (M+1)⁺.

### Example 12

### Cyclopentanecarboxylic acid (6-phenyl-furo[2,3-d]pyrimidin-4-yl)-amide

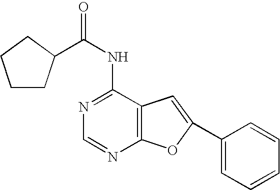

### Example 12

To a cooled solution of 6-Phenyl-furo[2,3-d]pyrimidin-4-ylamine (Intermediate 4, 42.2 mg, 0.20 mmol) in DMF (2.1 ml) was added sodium hydride (7.2 mg, 0.30 mmol) followed by cyclopenanecarbonyl chloride (27.7 μl, 0.22 mmol). The mixture was stirred at room temperature for 15 hours, and then poured into large amount of saturated ammonium chloride and the resulting solid collected by filtration. This material was purified by chromatography on silica gel eluting with chloroform to afford the title compound as a pale yellow solid (16%). 1H NMR (400 MHz, DMSO-d6) ppm 1.54-1.63(m, 2H), 1.66-1.85 (m, 4H), 1.91-1.97(m, 2H), 3.09(dt, J=8.0 Hz, 15.9 Hz), 7.44-7.48 (m, 1H), 7.52-7.56 (m, 3H), 7.92-7,94(m, 2H), 8.65 (s, 1H), 11.11(s, 1H). LC/MS: m/z 308 (M+1)⁺, 309 (M+2)⁺, 310 (M+3)⁺, 306 (M−1)⁻, 307 (M+1)⁻, 308 (M+2)⁻.

### Example 13

### Cyclopropanecarboxylic acid (6-phenyl-furo[2,3-d]pyrimidin-4-yl)-amide

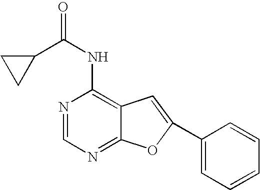

### Example 13

Prepared according to the similar methods as for Example 18.

1H NMR (400 MHz, DMSO-d6) ppm 0.91-1.00(m, 4H), 2.13-2.19(m, 1H), 7.56-7.61(m, 3H), 7.90-7.96(m, 2H), 8.67(s, 1H), 11.49(s, 1H). LC/MS: m/z 280 (M+1)⁺, 278 (M−1)⁻, 279 (M+1)⁻.

Intermediate 5

### 6-(4-Chloro-phenyl)-furo[2,3-d]pyrimidin-4-ylamine

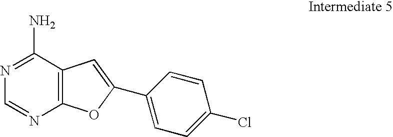

Prepared according to the similar methods as for Intermediate 3

1H NMR (400 MHz, CDCl3) ppm 5.20(br, 2H), 6.86(s, 1H), 7.43(m, 2H), 7.77(m, 2H), 8.40(s, 1H). LC/MS: m/z 246 (M+1)⁺.

### Example 14

### Cyclopentanecarboxylic acid [6-(4-chloro-phenyl)-furo[2,3-d]pyrimidin-4-yl]-amide

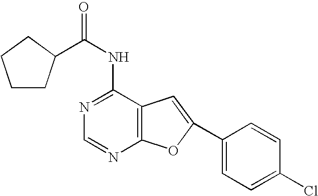

### Example 14

Prepared according to the similar methods as for Example 12.

1H NMR (400 MHz, DMSO-d6) ppm 1.54-1.64(m, 2H), 1.66-1.89(m, 4H), 1.91-1.99(m, 2H), 3.09(dt, J=8.0 Hz, 15.9 Hz ), 7.57-7.60(m, 3H), 7.94-7,97(m, 2H), 8.67(s, 1H), 11.14(s, 1H). LC/MS: m/z 342 (M+1)⁺, 344 (M+3)⁺, 345 (M+4)⁺, 340 (M−1)⁻, 342 (M+3)⁻, 343 (M+4)⁻. 820

### Example 15

### Cyclopropanecarboxylic acid [6-(4-chloro-phenyl)-furo[2,3-d]pyrimidin-4-yl]-amide

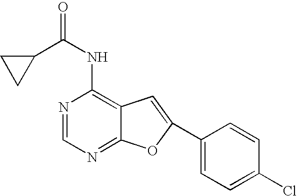

### Example 15

Prepared according to the similar methods as for Example 12.

1H NMR (400 MHz, DMSO-d6) ppm 0.91-0.99(m, 4H), 2.12-2.19(m, 1H), 7.44-7.58(m, 4H), 7.88-7.92(m, 2H), 8.67(s, 1H), 11.47(s, 1H). LC/MS: m/z 314 (M+1)⁺, 316 (M+3)⁺, 317 (M+4)⁺, 312 (M−1)−, 314 (M+1)−, 315 (M−2)³¹ .

Intermediate 6:

### 6-p-Tolyl-furo[2,3-d]pyrimidin-4-ylamine

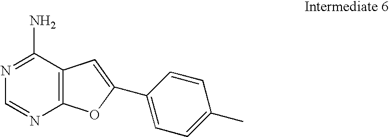

Prepared according to the similar methods as for Intermediate 3

1H NMR (400 MHz, CDCl3) ppm 2.42(s, 3H), 5.18(br, 2H), 6.81 (s, 1H), 7.28(m, 2H), 7.74(m, 2H), 8.39(s, 1H). LC/MS: m/z 226 (M+1)⁺.

### Example 16

### Cyclopentanecarboxylic acid (6-p-tolyl-furo[2,3-d]pyrimidin-4-yl)-amide

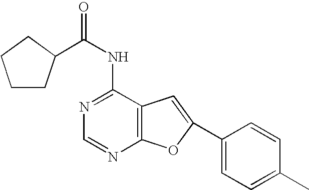

### Example 16

Prepared according to the similar methods as for Example 12.

1H NMR (400 MHz, DMSO-d6) ppm 1.57-1.61(m, 2H), 1.66-1.84(m, 4H), 1.90-1.97(m, 2H), 2.38(s, 3H), 3.08(dt, J=7.8 Hz, 16.2 Hz), 7.34 (d, J=8.1 Hz, 1H), 7.45(s, 1H), 7.81(d, J=8.1 Hz, 2H), 8.64(s, 1H), 11.09(s, 1H). LC/MS: m/z 322 (M+1)⁺, 323 (M+2)⁺, 324 (M+3)⁺, 320 (M−1)³¹ .

### Example 17

### Cyclopropanecarboxylic acid (6-p-tolyl-furo[2,3-d]pyrimidin-4-yl)-amide

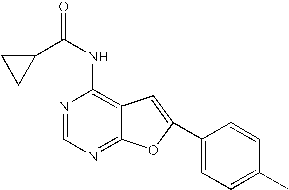

### Example 17

Prepared according to the similar methods as for Example 12.

1H NMR (400 MHz, DMSO-d6) ppm 0.91-0.99(m, 4H), 2.12-2.19(m, 1H), 2.37(s, 3H), 7.34(d, J=8.2 Hz, 2H), 7.44(s, 1H), 7.79(d, J=8.2 Hz, 2H), 8.64(s, 1H), 11.44(s, 1H). LC/MS: m/z 294 (M+1)⁺, 295 (M+2)⁺, 292 (M−1)−, 293 (M+1)³¹ .

Intermediate 7:

### 6-(4-Fluoro-phenyl)-furo[2,3-d]pyrimidin-4-ylamine

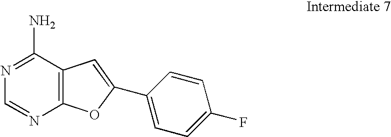

Prepared according to the similar methods as for Intermediate 3

1H NMR (400 MHz, DMSO-d6) ppm 5.22(br, 2H), 6.80(s, 1H), 7.17(m, 2H), 7.82(m, 2H), 8.39(s, 1H). LC/MS: m/z 230 (M+1)⁺.

### Example 18

### Cyclopentanecarboxylic acid [6-(4-fluoro-phenyl)-furo[2,3-d]pyrimidin-4-yl]-amide

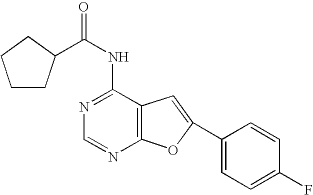

### Exampie 18

Prepared according to the similar methods as for Example 18

1H NMR (400 MHz, DMSO-d6) ppm 1.57-1.63(m, 2H), 1.66-1.85(m, 4H), 1.90-1.97(m, 2H), 3.09(dt, J=7.6 Hz, 15.9 Hz), 7.34-7.40 (m, 2H), 7.51(s, 1H), 7.98-8.01(m, 2H), 8.65 (s, 1H), 11.11(s, 1H). LC/MS: m/z 326 (M+1)⁺, 327 (M+2)⁺, 328 (M+3)⁺, 324 (M−1)−, 325 (M+1)−, 326 (M+2)³¹ .

### Example 19

### Cyclopropanecarboxylic acid [6-(4-fluoro-phenyl)-furo[2,3-d]pyrimidin-4-yl]-amide

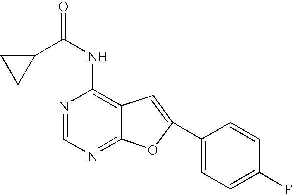

### Example 19

Prepared according to the similar methods as for Example 12.

1H NMR (400 MHz, DMSO-d6) ppm 0.91-0.97(m, 4H), 2.12-2.17(m, 1H), 7.34-7.39(m, 2H), 7.50(s, 1H), 7.95-7.98(m, 2H), 8.66(s, 1H), 11.46(s, 1H). LC/MS: m/z 298 (M+1)⁺, 299 (M+2)⁺, 300 (M+3)⁺, 296 (M−1)−, 297 (M+1)−, 298(M+2)³¹ . 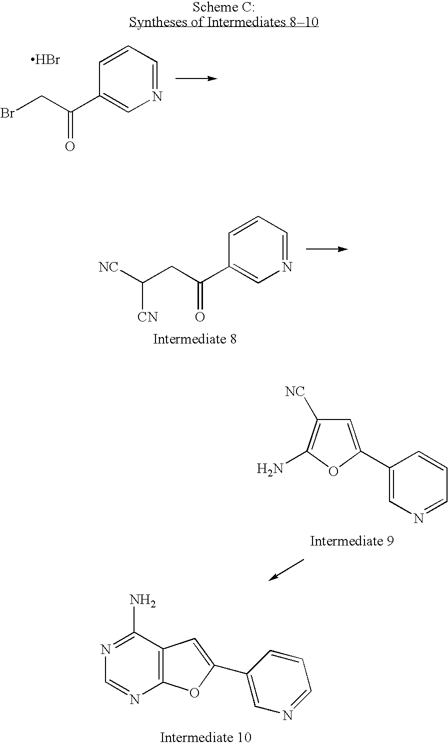
Intermediate 8

### 2-(2-Oxo-2-pyridin-3-yl-ethyl)-malononitrile

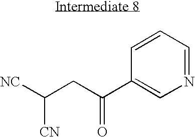

Prepared according to the similar methods as for Intermediate 1

1H NMR (400 MHz, CDCl3) ppm 3.79(d, J=6.8 Hz, 2H), 4.42(t, J=6.8 Hz, 1H), 7.52(m, 1H), 8.27(m, 1H), 8.90(m, 1H), 9.18(m, 1H).

Intermediate 9

### 2-Amino-5-pyridin-3-yl-furan-3-carbonitrile

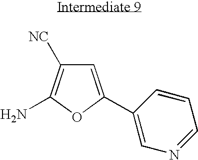

To a solution of 2-(2-oxo-2-pyridin-3-yl-ethyl)-malononitrile (Intermediate 8, 680 mg) in EtOH (30 ml) was added piperizine (2 ml). The mixture was stirred at room temperature for 30 min, and then concentrated in vaccuo. The residual oil was purified by chromatography on silica gel eluting with ethyl acetate to give the title compound as an orange solid (500 mg). TLC: Rf value=0.43 (eluting with ethyl acetate).

Intermediate 10

### 6-Pyridin-3-yl-furo[2,3-d]pyrimidin-4-ylamine

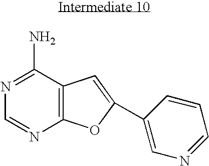

To a solution of (Intermediate 9, 500 mg) in AcOH (7 ml) was added diethoxymethyl acetate (0.9 ml). The mixture was stirred at room temperature for 1 hour, and then concentrated in vaccuo. The residual oil diluted with ethyl acetate was washed successively with sodium bicarbonate and brine, dried over magnesium sulfate, and concentrated in vaccuo to give a brown solid (527 mg). TLC: Rf value=0.57 (eluting with ethyl acetate). Thus obtained solid was suspended in the mixture of EtOH and THF (1:1, 40 ml). To the suspension with cooling in an ice-water bath was bubbled with NH3 gas for 30 min. The mixture was stirred at room temperature for 3 hours, concentrated in vaccuo, and suspended in the mixture of EtOH and THF (1:1, 32 ml). To the reaction mixture was added sodium ethoxide (6 ml of 0.46M in dry EtOH), stirred at room temperature for 2 hours, and concentrated in vaccuo to have a solution (ca 3 ml). The mixture was diluted with ethyl acetate, washed with sodium bicarbonate and brine, dried over magnesium sulfate, and concentrated in vaccuo. The residue was purified by preparative TLC (eluted with chloroform-MeOH) to afford the title compound as an orange colored solid (40 mg). 1H NMR (400 MHz, CDCl3) ppm 5.29(br, 2H), 7.00(s, 1H), 7.41(m, 1H), 8.13(m, 1H), 8.42(s, 1H), 8.62(m, 1H), 9.08(s, 1H). LC/MS: m/z 213 (M+1)⁺.

Intermediate 10 can also be made as follows.

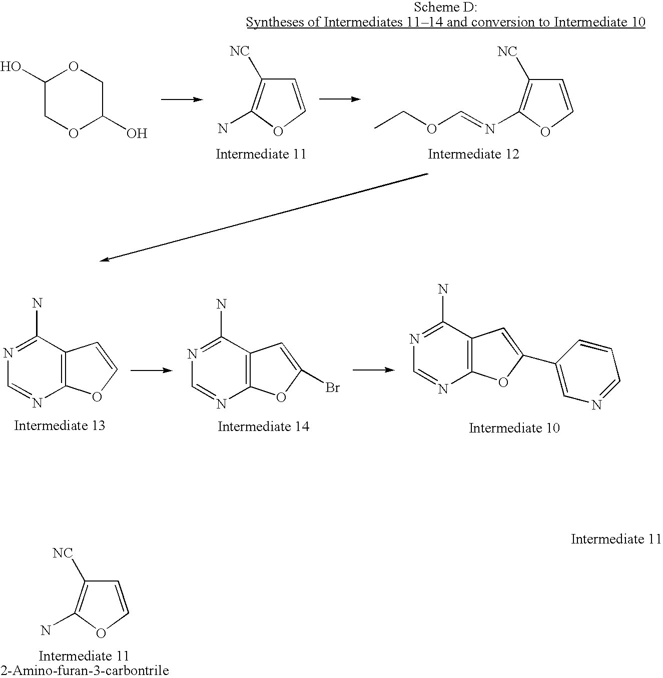

To a solution of 2,5-dihydroxy-1,4-dioxane (15.0 g, 125 mmol) in water (150 ml) was added 0.1N HCl (17 ml). To the mixture after stirred at room temperature over night was added malononitrile (14.87 g, 225 mmol) followed by diethylamine (23.3 ml, 225 mmol). The mixture was stirred at room temperature for 2 hours. To the mixture was added sodium bicarbonate (16 g), stirred at room temperature for 10 min, and extracted with ethyl acetate (3×300 ml). The combined organic layers were concentrated in vacuo. The residue was purified by chromatography on a short silica gel column (200 g silica gel) eluting with ethyl acetate to give the title compound as a pale yellow paste (15.27 g). This. crude compound still includes impurity by detecting TLC (developing with hexane-ethyl acetate 3:1) at the base line. 1H NMR (400 MHz, CDCl3) ppm 4.67 (br, 2H), 6.33(d, J=2.3 Hz, 1H), 6.77(d, J=2.3 Hz, 1H).

Intermediate 12

### N-(3-Cyano-furan-2-yl)-formimidic acid ethyl ester

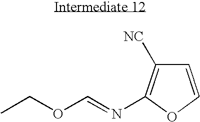

To a solution of 2-Amino-furan-3-carbonitrile (Intermediate 11, 15.27 g) in dioxane (200 ml) was added diethoxymethyl acetate (30 ml). The mixture after stirring at room temperature over night was added diethoxymethyl acetate (7 ml) then stirred one more night. The reaction mixture was diluted with toluene, treated with powder of sodium bicarbonate (70 g), and vigorously stirred for 20 min. Insoluble salts were removed by filtration and filtrate was concentrated in cacuo. The residual red oil was purified by chromatography on a silica gel column eluting with hexane-ethyl acetate (8:1) to give the title compound as white solids (9.57 g). 1H NMR (400 MHz, CDCl3) ppm 1.40 (t, J=7.2 Hz, 3H), 4.42 (q, J=7.2 Hz, 2H), 6.50 (d, J=2.3 Hz, 1H), 7.08 (d, J=2.3 Hz, 1H), 8.32 (S, 1H).

Intermediate 13

### Furo[2,3-d]pyrimidin-4-ylamine

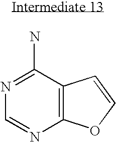

N-(3-Cyano-furan-2-yl)-formimidic acid ethyl ester (intermediate 12, 4.49 g) was dissolved in the mixture of EtOH and THF (1:1, 280 ml). To the solution under cooling in an ice-water bath was bubbled with NH3 gas for 1 hour. The cooling bath was removed and the mixture was stirred at room temperature for 4 hours. The mixture was concentrated in vacuo and dried under reduced pressure to afford the title compound as yellow solids (4.01 g). 1H NMR (400 MHz, CDCl3) ppm 5.20 (br, 2H), 6.67(d, J=2.5 Hz, 1H), 7.54(d, J=2.5 Hz, 1H), 8.41 (s, 1H). LC/MS: m/z 136 (M+1)⁺;

Intermediate 14

### 6-Bromo-furo[2,3-d]pyrimidin-4-ylamine

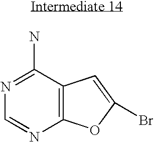

A solution of furo[2,3-d]pyrimidin-4-ylamine (Intermediate 13, 1.16 g) in THF (100 ml) was added dropwise to LDA in THF (0.71M, 30 ml, 21.5 mmol) at −78 ° C., and the mixture was stirred at this temperature for 30 min. 1,2-Dibromo-1,1,2,2-tetrafluoroethane (3.1 ml) was added to the mixture at −78° C. and stirred at −78° C. for 45 min. The reaction mixture was poured into a saturated solution of ammonium chloride and extracted with EtOAc. The organic layer was washed with brine, dried over Na₂SO₄, and concentrated in vacuo. The residue was purified by chromatography on a silica gel column eluting with CH₂Cl₂/acetone gradient (0˜50% acetone) to give the title compound as solids (1.00 g). 1H NMR (400 MHz, CDCl3) ppm 5.20 (br, 2H), 6.63(s, 1H), 8.35(s, 1H). LC/MS: m/z 214 (M)⁺, 216 (M+2)⁺;

Intermediate 10

### 6-Pyridin-3-yl-furo[2,3-d]pyrimidin-4-ylamine

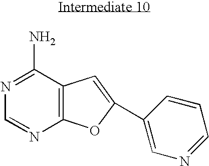

All the mixture of 6-bromo-furo[2,3-d]pyrimidin-4-ylamine (Intermediate 14, 1.0 g, 4.67 mmol), 3-pyridine boronic acid 1,3-propanediol cyclic ester (0.99 g, 6.07 mmol), tetrakis(triphenylphosphine)palladium(0) (432 mg, 0.37 mmol), K₃PO₄ (1.98 g, 9.34 mmol) was suspended in a mixture of DMF (50 ml) and water (12.5 ml). Under argon atmosphere the mixture was stirred on a heating bath (80° C.). The reaction mixture was reversed to room temperature then diluted with ethyl acetate (300 ml) and 5% aqueous sodium bicarbonate (170 ml). The aqueous phase was successively extracted with ethyl acetate (4×300 ml). Combined organic phase was washed with 5% aqueous sodium bicarbonate (170 ml), dried over magnesium sulfate, and concentrated in vacuo to give solids, which was suspended in small amount of ethyl acetate. The precipitated material was collected by filtration and dried under reduced pressure to give the title compound as pale yellow solids (880 mg). 1H NMR (400 MHz, CDCl3) ppm 5.29(br, 2H), 7.00(s, 1H), 7.41(m, 1H), 8.13(m, 1H), 8.42(s, 1H), 8.62(m, 1H), 9.08(s, 1H). LC/MS: m/z 213 (M+1)⁺.

### Example 20

### Cyclopentanecarboxylic acid (6-pyridin-3-yl-furo[2,3-d]pyrimidin-4-yl)-amide

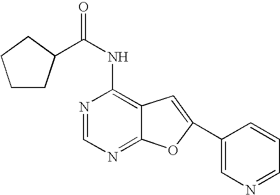

### Example 20

To a cooled solution of 6-pyridin-3-yl-furo[2,3-d]pyrimidin-4-ylamine (Intermediate 10, 33 mg, 0.16 mmol) in DMF (1.7 ml) was added sodium hydride (5.6 mg, 0.23 mmol) followed by cyclopenanecarbonyl chloride (23.5 μl, 0.19 mmol). The mixture was stirred at room temperature for 15 hours, and then poured into saturated ammonium chloride (10 ml) and extracted four times with chloroform (3 ml). The combined organic layers were washed with brine (20ml), and then concentrated under reduced pressure. The residue was purified by chromatography on silica gel eluting with chloroform-ethyl acetate gradient (0-50%) to give the title compound as a pale yellow solid (21%). 1H NMR (400 MHz, DMSO-d6) ppm 1.55-1.63(m, 2H), 1.66-1.84(m, 4H), 1.90-1.98(m, 2H), 3.09(dt, J=8.1 Hz, 15.9 Hz), 7.54-7.58(m, 1H), 7.70(s, 1H), 8.31-8.35(m, 1H), 8.63-8.65 (m, 2H), 9.14(m, 1H), 11.19(s, 1H). LC/MS: m/z 309 (M+1)⁺, 309 (M+1)⁺, 310 (M+2)⁺, 311 (M+3)⁺, 307 (M−1)−, 308 (M+1)−, 309 (M+2)³¹ .

### Example 21

### Cyclopropanecarboxylic acid (6-pyridin-3-yl-furo[2,3-d]pyrimidin-4-yl)-amide

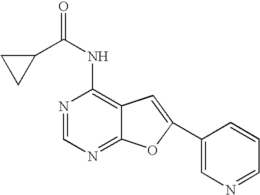

### Example 21

Prepared according to the similar methods as for Example 20.

1H NMR (400 MHz, DMSO-d6) ppm 0.92-0.99(m, 4H), 2.13-2.19(m, 1H), 7.54-7.57(m, 1H), 7.68(s, 1H), 8.29-8.33(m, 1H), 8.63(m, 1H), 8.64(s, 1H), 9.12(m, 1H), 11.54(s, 1H). LC/MS: m/z 281 (M+1)⁺, 282 (M+2)⁺, 283 (M+3)⁺, 279 (M−1)−, 280 (M)−, 281 (M+2)³¹ .

### Example 22

### Morpholine-4-carboxylic acid [6-(4-methoxy-phenyl)-furot[2,3d]pyrimidin-4-yl]-amide

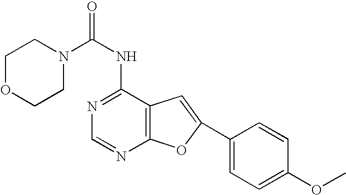

### Example 22

1H NMR (400 MHz, DMSO-d6) ppm 3.53-3.55(m, 4H), 3.64-3.67(m, 4H), 3.83(s, 3H), 7.06-7.13 (m, 3H), 7.82-7.86 (m, 2H), 8.54 (s, 1H), 10.18(s, 1H). LC/MS: m/z 355 (M+1)⁺, 356 (M+2)⁺, 357 (M+3)+,353 (M−1)−, 354 (M+1)−,356 (M+2)⁻.

### Example 23

### Pyrrolidine-1-carboxylic acid [6-(4-methoxy-phenyl)-furo[2,3-d]pyrimidin-4-yl]-amide

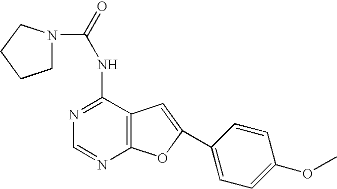

### Example 23

1H NMR (400 MHz, DMSO-d6) ppm 1.89(br, 4H), 3.51(br, 4H), 3.83(s, 3H), 7.07-7.09 (m, 2H), 7.23(s, 1H), 7.81-7.84 (m, 2H), 8.53 (s, 1H), 9.72(s, 1H). LC/MS: m/z 339 (M+1)⁺, 340 (M+2)⁺, 341 (M+3)⁺.

Biological Data

The compounds of the present invention have valuable pharmacologic properties. As described in more detail below, each of the compounds were tested for activity as GSK-3 inhibitors.

The protocol used to demonstrate the pharmacological response of the present invention is based on the ability of the kinase to phosphorylate a biotinylated peptide, the sequence of which is derived from the phosphorylation site of glycogen synthase and its sequence is: Biotin-Ahx-AAAKRREILSRRPS(PO₃)YR-amide. The phosphorylated biotinylated peptide is then captured onto streptavidin coated scintillation proximity assay (SPA) beads from Amersham Technology, where the signal from the ³³P is amplified via the scintillant contained in the beads.

GSK-3β, is commercially available or may be cloned and expressed in E coli using standard techniques to produce soluble, active protein. The production of active protein involves purification in two steps using Metal Chelate and Ion Exchange Chromatography. Protein eluting from Ion Exchange provides >90% pure product that may then be concentrated for use in high throughput screening.

The kinase was assayed at a concentration of 20 nM final in 100 mM HEPES, pH 7.2 containing 10 mM magnesium chloride, 0.1 mg/mL bovine serum albumin, 1 mM dithiothreitol, 0.3 mg/mL heparin, 2.8 uM peptide substrate, 2.5 uM ATP, and 0.2 uCi/well γ³³P]-ATP. After 40 minutes incubation at room temperature, the reaction was stopped by addition of 100 mM EDTA and 1 mM solution in 100 mM HEPES, pH7.2 followed by an additional solution of diluted Streptavidin coated SPA beads in PBS, pH 7.2 to give a final concentration of 0.25 mg of beads per assay well in a 96-well microtiter plate.

10 mM stock solutions of the compounds of the invention in 100% DMSO are generated as a first step in the screening process. The second step involves the creation of dose response plates where these compounds are diluted 10-fold in 100% DMSO to 1 mM concentrations and subsequently serially diluted 3-fold in 100% DMSO across the plate by automated liquid handling such that the final top concentration of inhibitor is 0.033 mM in the 30 uL kinase assay. The third step involves the creation of the assay plates. This is achieved by transferring 1 uL of the compounds to assay plates by automated liquid handling. The fourth step is to perform the assay as described and count the resulting plates in the Packard TopCount NXT microplate scintillation and luminescence counter. The final step is data acquisition and analysis where IC₅₀ values are generated for each compound by normalizing curve data to the equation 100*(U1−C2)/(C1−C2) (where U1 is the cpm value, C2 is the background, and C1 is the maximum number of counts), then fitting the normalized data to the equation y=Vmax*(1−(x/(K+x))). The IC₅₀ values were converted to pIC₅₀ values, i.e., -log IC₅₀ in Molar concentration.

Representative pIC₅₀ values for the compounds of the present invention are given in Table I.

TABLE I

| Example No Compounds | GSK-3 inhibition |
|---|---|
| 3 | +++ |
| 4 | ++ |
| 6 | + |
| 20 | ++++ |

+ = pIC₅₀ of <6.0;
++ = pIC₅₀ of 6.0-7.0;
+++ = pIC₅₀ of 7.0-8.0;
++++ = pIC₅₀ of >8.0;

Utility of the Present Invention

The above biological data clearly shows that the compounds of formula I are useful for treating or preventing GSK3 mediated diseases or conditions including, without limitation, diabetes, obesity, various CNS disorders such as Alzheimer's Disease, bipolar disorder, and schizophrenia, neurotraumatic injuries such as acute stroke, immune potentiation, baldness or hair loss, atherosclerotic cardiovascular disease, hypertension, polycystic ovary syndrome, ischemia, brain trauma or injury, immunodeficiency, and cancer.

1. A compound of the formula I, or a salt or solvate of: 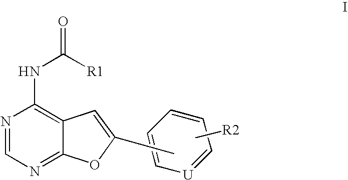
in which
U is CH or N; and
R1 is C₁₋₆alkyl, C₃₋₈cycloalkyl, —CH₂CH₂SCH₃, —CH₂—C₃₋₈cycloalkyl, phenyl optionally substituted with halogen or nitro; or
R1 is a radical of formula 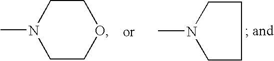
when U is CH, R2 is hydrogen, halogen, C₁₋₆alkyl, or —OCH₃; and
when U is N, R2 is hydrogen.

2. A method for the treatment or prophylaxis of a disorder in a mammal, said disorder being characterized by misregulation of GSK-3, comprising, administering to the mammal a therapeutically effective amount of a compound of the formula I of claim 1 or a salt, or solvate thereof.

3. The disorder of claim 2 that is selected from the list consisting of diabetes, obesity, Alzheimer's Disease, bipolar disorder, schizophrenia, stroke, baldness, hair loss, atherosclerotic cardiovascular disease, hypertension, polycystic ovary syndrome, ischemia, immunodeficiency, and cancer.

4. A pharmaceutical composition comprising a therapeutically effective amount of a compound of formula I of claim 1, or a salt or solvate thereof and one or more of pharmaceutically acceptable carriers, diluents and excipients.

5. A method of treating Type II Diabetes in a mammal, said method comprising administering to said mammal a therapeutically effective amount of a compound of formula I of claim 1, or salt or solvate or thereof.

6. A compound of formula I as claimed in claim 1, wherein said compound is selected from the group consisting of
Hexanoic acid [6-(4-methoxy-phenyl)-furo[2,3-d]pyrimidin-4-yl]-amide;
N-[6-(4-Methoxy-phenyl)-furo[2,3-d]pyrimidin-4-yl]-isobutyramide;
Cyclopentanecarboxylic acid [6-(4-methoxy-phenyl)-furo[2,3-d]pyrimidin-4-yl]-amide;
N-[6-(4-Methoxy-phenyl)-furo[2,3-d]pyrimidin-4-yl]-3-methylsulfanyl-propionamide;
3-Fluoro-N-[6-(4-methoxy-phenyl)-furo[2,3-d]pyrimidin-4-yl]-benzamide;
Cyclohexanecarboxylic acid [6-(4-methoxy-phenyl)-furo[2,3-d]pyrimidin-4-yl]-amide;
Cyclopropanecarboxylic acid [6-(4-methoxy-phenyl)-furo[2,3-d]pyrimidin-4-yl]-amide;
Furan-2-carboxylic acid [6-(4-methoxy-phenyl)-furo[2,3-d]pyrimidin-4-yl]-amide;
2-Cyclopentyl-N-[6-(4-methoxy-phenyl)-furo[2,3-d]pyrimidin-4-yl]-acetamide;
N-[6-(4-Methoxy-phenyl)-furo[2,3-d]pyrimidin-4-yl]-3-nitro-benzamide;
N-[6-(4-Methoxy-phenyl)-furo[2,3-d]pyrimidin-4-yl-4-nitro-benzamide;
Cyclopentanecarboxylic acid (6-phenyl-furo[2,3-d]pyrimidin-4-yl)-amide;
Cyclopropanecarboxylic acid (6-phenyl-furo[2,3-d]pyrimidin-4-yl)-amide;
Cyclopentanecarboxylic acid [6-(4-chloro-phenyl)-furo[2,3-d]pyrimidin-4-yl]-amide;
Cyclopropanecarboxylic acid [6-(4-chloro-phenyl)-furo[2,3-d]pyrimidin-4-yl]-amide;
Cyclopentanecarboxylic acid (6-p-tolyl-furo[2,3-d]pyrimidin-4-yl)-amide;
Cyclopropanecarboxylic acid (6-p-tolyl-furo[2,3-d]pyrimidin-4-yl)-amide;
Cyclopentanecarboxylic acid [6-(4-fluoro-phenyl)-furo[2,3-d]pyrimidin4-yl]-amide;
Cyclopropanecarboxylic acid [6-(4-fluoro-phenyl)-furo[2,3-d]pyrimidin-4-yl]-amide;
Cyclopentanecarboxylic acid (6-pyridin-3-yl-furo[2,3-d]pyrimidin4-yl)-amide; and
Cyclopropanecarboxylic acid (6-pyridin-3-yl-furo[2,3-d]pyrimidin-4-yl)-amide;
Morpholine-4-carboxylic acid [6-(4-methoxy-phenyl)-furo[2,3-d]pyrimidin-4-yl]-amide; and
Pyrrolidine-1-carboxylic acid [6-(4-methoxy-phenyl)-furo[2,3-d]pyrimidin-4-y]-amide.

7. A compound of formula I or salt or solvate thereof as claimed in claim 1, wherein said compound has the following formula: 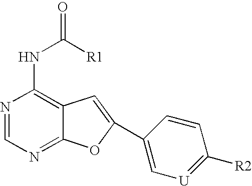
wherein
U is CH or N; and
R1 is C₁₋₆alkyl, C₃₋₈cycloalkyl, —CH₂CH₂SCH₃, —CH₂—C₃₋₈cycloalkyl, phenyl optionally substituted with halogen or nitro; or
R1 is a radical of formula 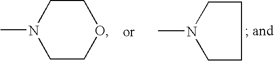
when U is CH, R2 is hydrogen, halogen, C₁₋₆alkyl, or —OCH₃; and
when U is N, R2 is hydrogen.
